(19) United States
(12) Patent Application Publication
(10) Pub. No.: US 2025/0367367 A1
(43) Pub. Date: Dec. 4, 2025

(54) Pump drive for a medicament delivery device

(71) Applicant: SHL Medical AG, Zug (CH)

(72) Inventors: Christoph Egloff, Zug (CH); Paolo Ravaynia, Zug (CH); Hsuan Wang, Zug (CH); Plamen Balkandjiev, Nacka Strand (SE); Stephen Allison Porter, Baltimore, MD (US); Mariano Mumpower, Baltimore, MD (US); Mason Watts, Batlimore, MD (US)

(21) Appl. No.: 18/874,135

(22) PCT Filed: May 31, 2023
(86) PCT No.: PCT/EP2023/064554

§ 371 Date: Dec. 12, 2024

Related U.S. Application Data
(60) Provisional application No. 63/351,851, filed on Jun. 14, 2022.
(60) Provisional application No. 63/400,772, filed on Aug. 25, 2022.

(30) Foreign Application Priority Data
Sep. 30, 2022 (EP) 22199275.3

(51) Int. Cl. A61M 5/142; A61M 5/172

(52) U.S. Cl. CPC A61M 5/14216; A61M 5/172; A61M 2205/0266; A61M 2205/3368

(57) ABSTRACT

The present disclosure provides a pump drive for a medicament delivery device. The pump drive comprises a pumping arrangement for conveying a medicament along a fluid path as part of a medicament delivery process. The pump drive further comprises a shape memory element configured to change shape according to a temperature thereof, wherein the shape memory element is operatively coupled to the pumping arrangement such that a change of shape of the shape memory element causes the pumping arrangement to convey the medicament along the fluid path. The pump drive further comprises a temperature control arrangement configured to change the temperature of the shape memory element and thereby change the shape of the shape memory element.

### CROSS-REFERENCE TO RELATED APPLICATIONS

The present application is a U.S. National Phase Application pursuant to 35 U.S.C. § 371 of International Application No. PCT/EP2023/064554 filed May 31, 2023, which claims priority to (i) U.S. Provisional Application No. 63/351,851 filed Jun. 14, 2022, (ii) U.S. Provisional Application No. 63/400,772 filed Aug. 25, 2022, and (iii) EP patent application Ser. No. 22/199,275.3 filed Sep. 30, 2022. The entire disclosure contents of these applications are herewith incorporated by reference into the present application.

### TECHNICAL FIELD

The present disclosure relates to a device for medicament delivery. More particularly, the present disclosure relates to a pump drive for a medicament delivery device and a medicament delivery device containing such a pump drive.

### BACKGROUND

In the administration of liquid formulations of pharmaceutical agents (also referred to as ‘medicaments’), it is often necessary to deliver well defined volumes of liquid. Medicaments are typically injected into the body of a patient. For parenteral injection, hypodermic syringes, drug pens or motor driven systems are employed.

In the case of medicaments which have to be administered over a length of time and/or according to a specified schedule, syringes and pens are increasingly being replaced by motor driven systems. Many motor driven systems exist to deliver medicaments, as in the case of parenteral delivery. For instance, motor driven liquid displacement pumps are common in the art.

Conventional liquid displacement pump units are mainly driven by electric motors, which have constant power consumption, are noisy, and contribute significant weight to medicament delivery devices.

Thus, there is a need for improved medicament delivery devices that overcome one or more of these limitations, and/or other limitations in prior art medicament delivery devices.

### SUMMARY

It is realized as a part of the present disclosure that electric motors of conventional medicament delivery devices are typically used to convey medicament (i.e., in fluid form) along predefined fluid paths, and thus are not controlled according to complex control schemes. Thus, a simplified arrangement may be provided for causing the pump drive to convey fluid along a fluid path.

In particular, according to an aspect of the present disclosure, there is provided a pump drive for a medicament delivery device, comprising a pumping arrangement for conveying a medicament along a fluid path as part of a medicament delivery process, and a shape memory element configured to change shape according to a temperature thereof, wherein the shape memory element is operatively coupled to the pumping arrangement such that a change of shape of the shape memory element causes the pumping arrangement to convey the medicament along the fluid path. Said pump drive further comprises a temperature control arrangement configured to change the temperature of the shape memory element and thereby change the shape of the shape memory element.

By using a shape memory element, which may be a shape-memory alloy such as nitinol, Co—Ni—Al, or Fe—Mn—Si, or another shape memory material, a pump drive may advantageously be provided that has significantly reduced weight, and that produces significantly less sound when operated. Moreover, such a pump drive may be manufactured without the use of printed circuit boards (PCBs) or other electronic components which may be radiation-sensitive. Thus, the pump drive may advantageously be sterilized using gamma radiation, making it especially suitable for applications in medicament delivery devices.

The pumping arrangement may comprise any suitable components for arranging in or around the fluid path and encouraging a conveyance of medicament along the fluid path. For example, the pumping arrangement may comprise a syringe arrangement, a peristaltic pump arrangement, or some other type of pump arrangement, depending on the implementation. In any event, an operation or motion of the pumping arrangement is able to encourage a conveyance of fluid along the fluid path.

The fluid path may be between a reservoir of medicament and a pumping (e.g., dosing) chamber, between a chamber and an outlet of the medicament delivery device, and/or between some other locations within or outside the medicament delivery device.

The shape memory element may be operatively coupled to the pumping arrangement in any suitable way such that a change of shape of the shape memory element may induce the pumping arrangement to convey medicament along the fluid path. For example, the shape memory element may be bonded or affixed to, wrapped, tied, or looped around, or otherwise coupled to a part of the pumping arrangement, such that the change of shape of the shape change element causes the pumping arrangement to operate to convey medicament along the fluid path.

The change of shape of the shape memory element may cause a pumping element of the pumping arrangement to move from a first state to a second state and/or from the second state to the first state. The pumping element may comprise a plunger or a membrane, for example. That is, a first state of the pumping element may comprise a plunged or depressed state of a plunger, and a second state may comprise an extended or withdrawn state of the plunger. By controlling the temperature of the shape memory element (e.g., heating and/or cooling), the pumping element may be moved between the first state and the second state.

In some example embodiments, the pumping arrangement may comprise a pumping chamber coupled to an inlet, a first motion of the pumping element from the first state to the second state may convey the medicament into the pumping chamber via the inlet. Additionally or alternatively, the pumping arrangement comprises a (same or second) pumping chamber coupled to an outlet, and a second motion of the pumping element from the second state to the first state may convey the medicament out of the pumping chamber via the outlet.

The pumping chamber may define a dosing volume for a medicament such that each discharge of the pumping chamber may define a dose, and may be timed or scheduled according to a treatment schedule for a patient.

Depending on the implementation, the pumping chamber may be coupled to the inlet and/or the outlet via one-way valves or electronically controlled valves. It may be preferred to reduce the number of electronic components, in which case mechanical one-way valves may be used. Alternatively, it may be preferred to increase the controllability of the pump drive, in which case electronically controlled valves may be used.

In some examples, the temperate control arrangement may be configured to repeatedly change the shape of the shape memory element between a first shape and a second shape, and repeatedly changing the shape of the shape memory element between the first shape and the second shape may thereby cause a reciprocal motion of a pumping element of the pump arrangement. For example, the aforementioned first and second motions of a pumping element may be repeated so as to achieve a continual conveyance of medicament along the fluid path.

In some example embodiments, the pumping arrangement may comprise a biasing element configured to bias the pumping element into the first state such that a motion (e.g., the first motion as referred to above) of the pumping element from the first state to the second state biases the pumping element against the biasing member. Therefore, a return motion (e.g., the second motion as referred to above) may be motivated by a biasing force applied by the biasing member, returning the pumping element to the first state. It will be appreciated that the biasing member may, in some other example embodiments, bias the pumping element into the second state such that a motion of the pumping element from the second state to the first state biases the pumping element against the biasing member.

Hence, according to such example embodiments, only a motion that biases against the biasing member requires power to carry out. The biasing member may then store energy (e.g., as elastic energy) and release said energy to perform the other motion of, e.g., the pumping element returning to the first (or second) state as a part of a repeated reciprocal motion. Therefore, a power consumption of the pump drive may be advantageously reduced.

It will be appreciated that the temperature control arrangement may be configured to change the temperature of the shape memory element away from an ambient temperature, or between two elevated temperatures, two reduced temperatures, or an elevated temperature and a reduced temperature (relative to ambient temperature). For example, the temperature control arrangement may comprise a heating arrangement and/or a cooling arrangement employing any suitable heating or cooling means, respectively.

The temperature control may exploit a temperature difference in an ambient environment, which may be as a result of a proximity of the pump drive to a source of heat (e.g., a body heat of a patient) or may be artificially induced, such that a motion of the shape memory element throughout the ambient environment may cause a change in temperature thereof.

In some preferred embodiments of the present disclosure, the shape memory element may be electrically conductive, and the temperature control arrangement may comprise one or more electrical contacts configured to pass an electrical current through the shape memory element to thereby change the temperature of the shape memory element. That is, Ohmic heating may be employed to cause the shape memory element to heat up when a current is passed through it, and then the shape memory element may be allowed to cool, such that the shape memory element changes shape accordingly.

Such an approach of using Ohmic heating may advantageously further improve the power consumption efficiency of the pump drive, as well as simplify the construction of the pump drive, as the shape memory element itself may act as effectively its own heater, with no efficiency losses caused by an inefficient transfer of heat between, for example, a heating arrangement and the shape memory element.

In some example embodiments, a change of shape of the shape memory element may causes the temperature control arrangement to be decoupled (i.e., thermally decoupled) from the shape memory element, in such a way that the temperature control arrangement no longer heats (or cools) the shape memory element. The decoupling of the temperature control arrangement from the shape memory element may cause the shape memory element to change shape as the shape memory element returns to an ambient temperature as a result of said thermal decoupling. This change of shape of the shape memory element caused by the decoupling may thereby recouple the temperature control arrangement to the shape memory element, i.e., as it returns to its original shape.

This process may then be caused to or allowed to repeat so as to achieve a reciprocal motion of the pumping arrangement, said pumping arrangement being operatively coupled to the shape memory element and induced to convey medicament along a fluid path according to a change of shape of the shape memory element.

Therefore, the control operation of the pump drive may be greatly simplified, and the control components (e.g., electronics) may be minimized or left out of the medicament delivery device. That is, the motion of the pump drive may be initiated in some way and then allowed to cause the reciprocal motion of the pumping arrangement under the action of the thermal coupling, decoupling, and recoupling of the temperature control arrangement to and from the shape memory element.

In some examples, the shape memory element is formed as a wire, but in other examples the shape memory element may be formed of substantially any shape such as a rectangle, a ring or band, a sphere, etc.

In the example wherein the shape memory element is formed as a wire, the change of shape of the shape memory element may comprise a change of length of the wire. In examples wherein the shape memory element has another shape, the change of shape of the shape memory element may comprise a change in the length, width, and/or height of the shape memory element, either in proportion with each other or out of proportion with each other. That is, the change in shape may be only along one dimension of a two- or three-dimensional shape, or only along two dimensions of a three-dimensional shape.

In some example embodiments, the shape memory element may be tensioned, and the tensioning may be facilitated by the use of a biasing member as described above, or using some other means. The shape memory element may be folded, wrapped, or wound around one or more tensioning elements so as to enhance the shape change effect of the shape memory element in a smaller form factor.

For example, if the shape memory element is a wire and said wire is wrapped around a tensioning element on its path from an anchor to a point of coupling with the pumping arrangement, then a proportional change in length (e.g., a shortening by 10%) of the wire may result in a greater magnitude of displacement of the ends of the wire towards each other, relative to a shorter initial shape memory element connected directly between said anchor and said point of coupling (i.e., without the extra winding). Therefore, the effect of the shape change of the shape memory element may be enhanced or fine tuned depending on the desired displacement caused by said shape change, and the form factor of the pump drive (and hence the overall medicament delivery device) may be reduced.

According to another aspect of the present disclosure, there is provided a medicament delivery device comprising the pump drive substantially as described above. The medicament delivery device may be configured for implantation into a patient, affixation (e.g., using adhesive pads or straps) onto a patient.

The medicament delivery device may further comprise control circuitry for controlling the pump drive, communication circuitry for communicating with a user interface, indicating circuitry for indicating a state of the medicament delivery device and/or other circuitry depending on the implementation.

The medicament delivery device may further comprise, in some examples, a reservoir for storing medicament, or the medicament may be provided from an external reservoir. The medicament delivery device may further comprise one or more medicament delivery mechanisms such as needles that are configured to deliver the medicament into a patient.

According to yet another aspect of the present disclosure, there is provided a method of operating the pump drive substantially as described above. The method comprises changing, using the temperature control arrangement, the temperature of the shape memory element to thereby cause the shape memory element to change shape and thereby cause the pumping arrangement to pump the medicament along the fluid path.

The method may be carried out by control circuitry of the medicament delivery device, or by external circuitry in communication with communication circuitry of the medicament delivery device, or the method may be substantially mechanically implemented (i.e., without control circuitry), depending on the implementation.

In any event, it will be appreciated that numerous advantages, some of which are discussed above, can be provided through the use of a shape memory element driving a pumping arrangement instead of an electric motor, as described herein according to aspects of the present disclosure. Various examples of embodiments of the present disclosure are discussed below.

### BRIEF DESCRIPTION OF THE DRAWINGS

One or more embodiments will be described, by way of example only, and with reference to the following figures, in which:

FIGS. 1A to 1C schematically show a pump drive for a medicament delivery device according to an embodiment of the present disclosure;

FIGS. 2A and 2B schematically show a pump drive for a medicament delivery device according to another embodiment of the present disclosure; and

FIG. 3 illustrates a method for of operating a pump drive according to an embodiment of the present disclosure.

### DETAILED DESCRIPTION

The present disclosure is described in the following by way of a number of illustrative examples. It will be appreciated that these examples are provided for illustration and explanation only and are not intended to be limiting on the scope of the disclosure. Moreover, where different individual embodiments are described, it will be appreciated that such embodiments, or at least parts thereof, may be combined.

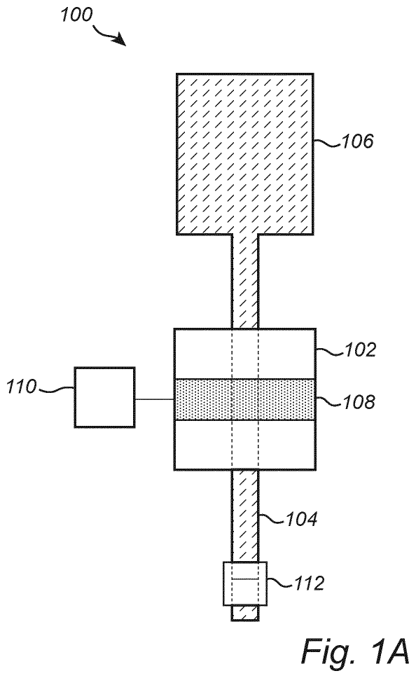

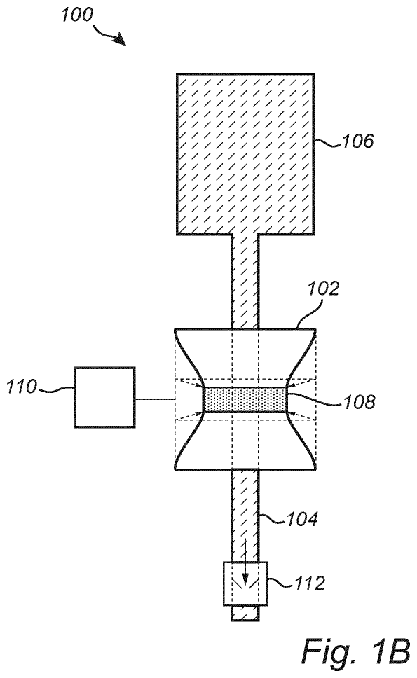

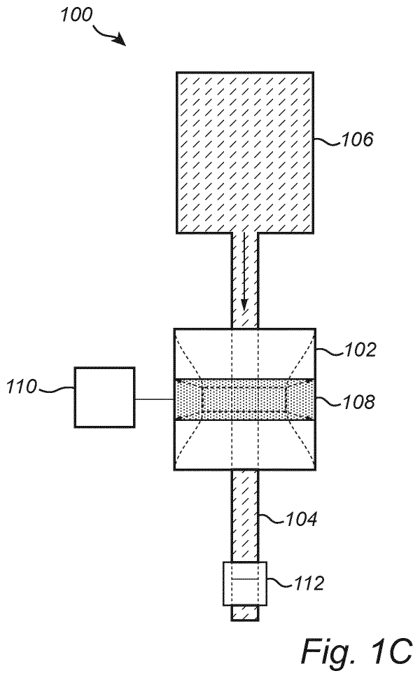

FIGS. 1A to 1C schematically show a pump drive 100 for a medicament delivery device (not shown) according to an embodiment of the present disclosure.

The pump drive 100 comprises a pumping arrangement 102 arranged to convey medicament along a fluid path 104, which in this illustrated example is from a medicament reservoir 106. The illustrated pumping arrangement 102 comprises a flexible membrane, shaped as a cylinder, and is arranged around the fluid path 104, which may be defined by tubing (e.g., plastic tubing), such that a restriction of the flexible membrane impinges upon the tubing and encourages a flow of medicament along the fluid path 104 much in the same way that a peristaltic pump may operate.

It will be appreciated that the illustrated embodiment may alternatively be configured such that pumping arrangement 102 defines an internal pumping chamber into which medicament can be drawn and out of which medicament can be expelled upon a restriction of the flexible membrane of the pumping arrangement 102 is formed. In any event, it will be appreciated that, in the illustrated embodiment, the pumping action of the pumping arrangement 102 is caused by the restriction of the flexible membrane.

Arranged around the pumping arrangement 102 is a shape memory element 108 formed as a band that encircles the flexible membrane of the pumping arrangement 102. The shape memory element 108 is conductive (e.g., made of a shape-memory alloy such as nitinol) and electrically connected to a temperature control arrangement 110.

The temperature control arrangement 110 may thus be operated, e.g., according to predefined instructions, instructions communicated to it, or otherwise, to control a current passing through the shape memory element 108. When current passes through the shape memory element 108, it is heated by Ohmic heating. Preferably, the temperature to which the shape memory element 108 is brought during operation of the pump drive 100 is kept below any temperature that could cause damage to other components of the pump drive 100.

The operation of the pump drive 100 is illustrated in FIGS. 1B and 1C. The shape memory element 108 may be initially shaped as part of a manufacturing process of the pump drive 100 so as to have a band shape defining a first radius. Thus, as shown in FIG. 1B, the temperature control arrangement 110, when allowing the shape memory element to adopt its original (i.e., ‘remembered’) shape—that is, when no current is passed through the shape memory element 108 and it cools (e.g., through conductive, convective and/or radiative cooling) to an ambient temperature—the band-shaped shape memory element may restrict the flexible membrane of the pumping arrangement 102 and thereby cause a flow of medicament along the fluid path 104 and through a one-way valve 112. Shape changes are indicated by dotted arrows, and fluid flow is indicated by the solid arrow.

As shown in FIG. 1C, when heated by the temperature control arrangement 110, the shape memory element 108 loses this shape and is biased by the elastic expansion of the flexible membrane of the pumping arrangement 102 to adopt a band shape defining a second radius larger than the first radius, i.e., corresponding to a radius of the cylindrical flexible membrane of the pumping arrangement 102, as indicated by the dotted arrows. This action of the pumping arrangement 102, in combination with the closing of the one-way valve 112, thereby causes more medicament to be drawn into the fluid path 104 from the reservoir 106, as shown by the solid arrow.

It will be appreciated that the restriction and release of the flexible membrane of the pumping arrangement 102 by the shape memory element 108, according to its temperature as controlled by the temperate control arrangement, may be repeated as many times as necessary to convey a desired amount of medicament along the fluid path 104.

In some example adaptations, a sensor may be introduced to measure the amount of medicament conveyed by the pumping arrangement. The sensor may measure a number of times the pumping arrangement 102 has been actuated, the number of times the current through the shape memory element 108 has been cycled (i.e., on and off), and/or the sensor may measure the fluid flow directly or indirectly. Such information may be relayed to a user, for example.

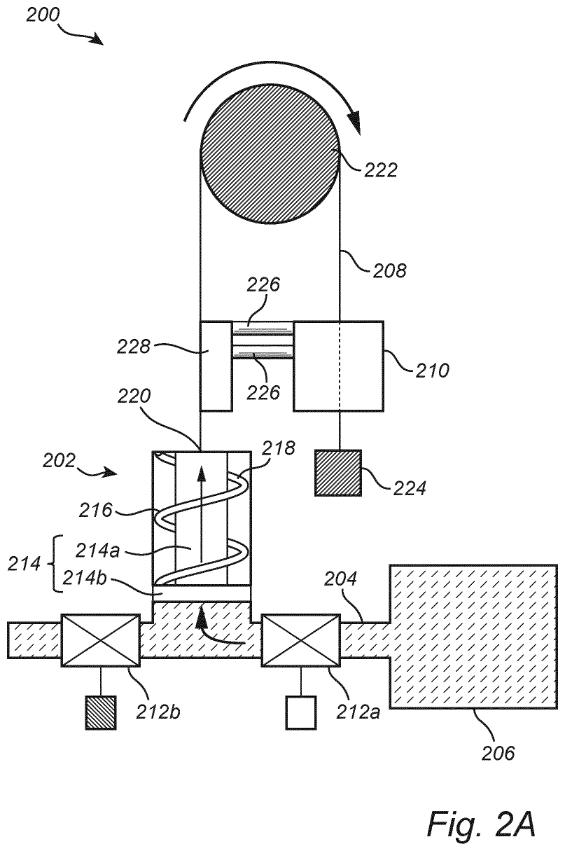

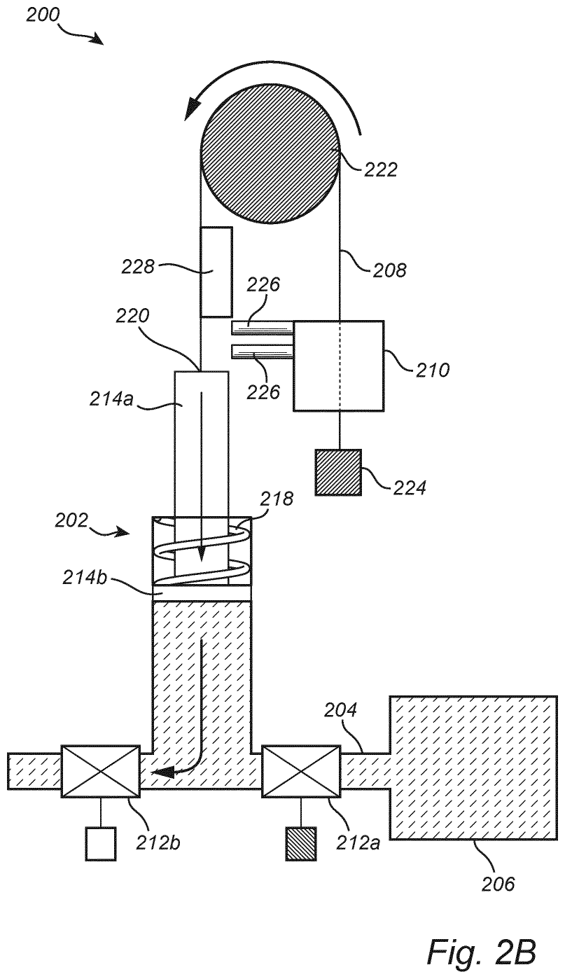

FIGS. 2A and 2B schematically show a pump drive 200 for a medicament delivery device (not shown) according to another embodiment of the present disclosure. Components shown in FIG. 2, and indicated by reference numerals having a value incremented by 100 relative to a reference number FIGS. 1A to 1C, may correspond to a same or similar element, at least in respect of intended function, as the component indicated by the corresponding reference numeral in FIGS. 1A to 1C.

The pump drive 200 comprises a pumping arrangement 202 arranged on a fluid path 104 and configured to convey medicament there along, from a medicament reservoir 206. The pumping arrangement 202 in this illustrated example is a syringe-type pump that employs a plunger 214 with a plunger shaft 214a and a plunger head 214b arranged in a pumping chamber 216.

As shown in FIGS. 2A and 2B, the pumping arrangement 202 further comprises a biasing element 218 arranged to bias the plunger 214 in a depressed state.

The pump drive 200 further comprises a shape memory element 208 formed as a wire, operatively coupled to the pumping arrangement 202 by affixation to the plunger shaft 214a at a coupling point 220. The shape memory element 208 extends from one end affixed to the coupling point 220, around a tensioning element 222, and to its other end affixed to an anchor 224. The tensioning element 222 and the anchor 224 may be fixed as non-moving parts of the pump drive 200. For example, such non-moving parts may be affixed to a housing or casing of a medicament delivery device (not shown) into which the pump drive 200 is installed.

The tensioning element 222 may comprise a bearing or may otherwise be configured to allow an unhindered (e.g., low friction) passage of the shape memory element 208 about its surface. The tensioning element 222 may be a metal cylinder, for example, which may have a groove or channel formed therein for receiving and guiding the wire-shaped shape memory element 208. In some other examples, the shape memory element 208 may pass through an opening formed in the tensioning element 222. Moreover, in some examples, there may be more than one tensioning element 222, depending on the implementation. Such one or more tensioning elements 222 may be arranged to retain and/or guide the shape memory element 208, especially during its change of shape, so that the shape memory element 208 can be routed unhindered through an internal of a medicament delivery device, which may preferably have a small form factor for arrangement on or in a patient.

The shape memory element 208 passes through a temperature control arrangement 210 which is configured to change the temperature of the shape memory element. The temperature control arrangement 210 in this example comprises an electric heater, and a pair of contacts 226 that, when interconnected by a bridging contact 228 arranged on the shape memory element 208, allow a powering on of the electric heater of the temperature control arrangement 210.

In FIG. 2A, the plunger 214 is shown in a depressed state, being biased theretoward by the biasing element 218. The bridging contact 228 interconnects the contacts 226 of the temperature control arrangement 210 and thus the heater therein is powered on, heating up the shape memory element 208.

The shape memory element 208, when heated, shrinks and thereby decreases the length of the wire between the anchor 224 and the coupling point 220 on the plunger shaft 214a, passing around the tensioning element 222. It is will be appreciated that the passage of the shape memory element around this tensioning element 222 enhances the shortening of the wire-shaped shape memory element 208 (e.g., by approximately two-fold). That is, the length of the shape memory element 208 is shortened by a proportion X, such that the length from the anchor 224 to the tensioning element 222, and the length from the tensioning element 222 to the coupling point 220, reduces by (1-X) L. Hence, a greater displacement of the plunger 214 is achieved relative to a comparative example wherein the anchor 224 is arranged in the position of the tensioning element 222.

When the plunger 214 is pulled upwards, as illustrated in FIG. 2A and indicated by the solid vertical arrow, the plunger head 214b abuts the biasing element 218, i.e., a spring coiled around the plunger shaft 214a, and thereby loads the biasing element 218 with elastic energy.

As the plunger is pulled upwards, a first electronically controlled valve 212a, acting as an inlet, is opened (as indicated by the white box) while a second electronically controlled valve 212b is closed (as indicated by the black box) and the first electronically controlled valve 212a allows medicament to be drawn from the reservoir 206 and into the pumping chamber 216.

Furthermore, the bridging contact 228 arranged on the shape memory element 208 is moved upward and therefore breaks the connection between the contacts 226 of the temperature control arrangement 210, causing the heater therein to be depowered and thus allowing the shape memory element 208 to cool to, e.g., an ambient temperature of the pump drive 200.

As shown in FIG. 2B, the cooling of the wire-shaped shape memory element 208 causes the length of the shape memory element to increase again. The shape memory element 208 is therefore guided back around the tensioning element 222 as the plunger 214 is pushed back into the depressed state by the action of the biasing element 218 acting against the plunger head 214b. During this time, it will be appreciated that, advantageously, no power is consumed by the temperature control arrangement 210.

As the plunger 214 is pushed downwards, the second electronically controlled valve 212b, acting as an outlet, is opened (as indicated by the white box) while the first electronically controlled valve 212a is closed (as indicated by the black box) and thus the second electronically controlled valve 212b allows medicament to be expelled from the pumping chamber 216. The medicament may then be delivered to a patient, for example. The size of the pumping chamber 216 may be configured according to a dosing size or a portion thereof, such that each (one or more) filling and emptying of the pumping chamber 216 corresponds to a dose of medicament for a patient.

Furthermore, as the plunger 214 is pushed downwards, the bridging contact 228 again bridges the contacts 226 of the temperature control arrangement 210. Thus, the electric heater in the temperature control arrangement 210 is powered on again, causing the shape memory element 208 to be heated again, and thus the pump drive 200 returns to the state as illustrated in FIG. 2A.

As with the operation described in respect of FIGS. 1A to 1C, the operation illustrated in FIGS. 2A and 2B may be repeated one or more times, causing a reciprocal motion of the pumping arrangement 202 (in particular, of the plunger).

The electronically controlled valves 212a, 212b may be controlled by onboard or an external control circuitry, or may be automatically controlled according to the motion of, e.g., the bridging contact 228, as with the powering on and off of the heater in the temperature control arrangement. In some examples, the pump drive 200 may comprise further modes of operation, comprising, for example, a closing or opening of both electronically controlled valves 212a, 212b.

It will be appreciated that various elements of each of the specifically described embodiments shown in FIGS. 1A to 1C and 2A and 2B may be interchanged, combined, or otherwise rearranged, without a loss of interoperability. The choice of construction and configuration for each component may be motivated by various factors such as size limitations, desire for improved controllability, desire for lower power consumption, etc.

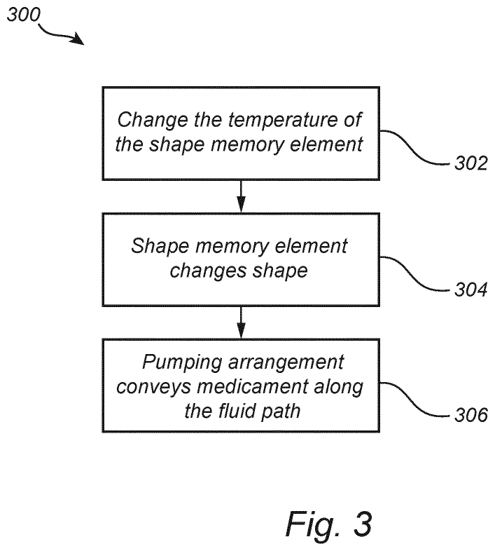

FIG. 3 illustrates a method 300 for of operating a pump drive such as the pump drives 100, 200 described above, according to an embodiment of the present disclosure.

The method 300 comprises changing the temperature of the shape memory element (step 302). As discussed above, this may be achieved by applying heat from a heater, cold from a cooler, applying an electrical current through a (electrically conductive) shape memory element, or by some other means.

By changing the temperature of the shape memory element, the shape memory element is caused to change shape (step 304). For example, the shape memory element may return to, or relax from, a predefined shape that is ‘remembered’ by the shape memory element.

As described above, the shape memory element is operatively coupled to a pumping arrangement such that the change of shape of the shape memory element (step 304) causes the pumping arrangement to convey medicament along the fluid path (step 306).

It will be appreciated that steps 304 and 306 may result from the performance of step 302. Thus, if and when a conveyance of medicament along the fluid path is desired, it may only be necessary to perform step 302 and change the temperature of the shape memory element, or initiate said changing of temperature, e.g., by powering on or off a heater, passing a current through the shape change element, etc., as discussed above.

The method 300 may be performed repeatedly depending on whether a single or continuous conveyance of medicament is desired. For example, the method 300 may be scheduled according to a dosing regime for a patient requiring such a scheduled delivery of the medicament.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, there can a one or more position sensors used in the device as disclosed herein for detecting the position of the plunger. For example, the nitinol pump is wrapped in a coil of conductive wire. The wire wrap has a variable pitch, changing from dense winding at one end to sparse winding at the other. The winding terminates along with the nitinol drive wires at the boundary of the pump module. The segment of the mechanism within the pump cylinder that moves with the reciprocating pump stroke cycle includes a piece of ferromagnetic material. The wire wrap forms a simple inductor, and the ferromagnetic material influences the inductance of the coil based on its position. The inductance of the coil is increased when the ferromagnetic material is adjacent to the densely wrapped segment of coil. The inductance of the coil is decreased when the ferromagnetic material is adjacent to the sparsely wrapped segment of coil.

While the present disclosure is susceptible to various modifications and alternative forms, specific embodiments are shown and described by way of example in relation to the drawings, with a view to clearly explaining the various advantageous aspects of the present disclosure. It should be understood, however, that the detailed description herein and the drawings attached hereto are not intended to limit the disclosure to the particular form disclosed. Rather, the intention is to cover all modifications, equivalents, and alternatives falling within the scope of the following claims.

1-15 (canceled)

16. A pump drive for a medicament delivery device, comprising:
a pumping arrangement for conveying a medicament along a fluid path as part of a medicament delivery process;
a shape memory element configured to change shape according to a temperature thereof, wherein the shape memory element is operatively coupled to the pumping arrangement such that a change of shape of the shape memory element causes the pumping arrangement to convey the medicament along the fluid path; and
a temperature control arrangement configured to change the temperature of the shape memory element and thereby change the shape of the shape memory element.

17. The pump drive according to claim 16, wherein:
the change of shape of the shape memory element causes a pumping element of the pumping arrangement to move from a first state to a second state and/or from the second state to the first state.

18. The pump drive according to claim 17, wherein:
the pumping arrangement comprises a pumping chamber coupled to an inlet; and
a first motion of the pumping element from the first state to the second state conveys the medicament into the pumping chamber via the inlet.

19. The pump drive according to claim 17, wherein:
the pumping arrangement comprises a pumping chamber coupled to an outlet; and
a second motion of the pumping element from the second state to the first state conveys the medicament out of the pumping chamber via the outlet.

20. The pump drive according to claim 19, wherein:
the pumping chamber is coupled to the inlet and/or the outlet via one-way valves or electronically controlled valves.

21. The pump drive according to claim 16, wherein:
the temperate control arrangement is configured to repeatedly change the shape of the shape memory element between a first shape and a second shape; and
repeatedly changing the shape of the shape memory element between the first shape and the second shape causes a reciprocal motion of a pumping element of the pump arrangement.

22. The pump drive according to claim 17, wherein:
the pumping element comprises a plunger or a membrane.

23. The pump drive according to claim 17, wherein:
the pumping arrangement comprises a biasing element biasing the pumping element into the first state such that a motion of the pumping element from the first state to the second state biases the pumping element against the biasing element.

24. The pump drive according to claim 16, wherein:
the shape memory element is electrically conductive; and
the temperature control arrangement comprises one or more electrical contacts configured to pass an electrical current through the shape memory element to thereby change the temperature of the shape memory element.

25. The pump drive according to claim 16, wherein:
a change of shape of the shape memory element causes the temperature control arrangement to be decoupled from the shape memory element; and
the decoupling of the temperature control arrangement from the shape memory element causes the shape memory element to change shape and thereby recouple the temperature control arrangement to the shape memory element.

26. The pump drive according to claim 16, wherein:
the shape memory element is formed as a wire.

27. The pump drive according to claim 26, wherein:
the change of shape of the shape memory element comprises a change of length of the wire.

28. The pump drive according to claim 16, wherein:
wherein the shape memory element comprises nitinol.

29. A medicament delivery device comprising the pump drive according to claim 16.

30. A method of operating the pump drive according to claim 16, comprising:
changing, using the temperature control arrangement, the temperature of the shape memory element to thereby cause the shape memory element to change shape and thereby cause the pumping arrangement to pump the medicament along the fluid path.
